(19) United States
(12) Patent Application Publication
(10) Pub. No.: US 2025/0352156 A1
(43) Pub. Date: Nov. 20, 2025

(54) METHOD FOR SPECIFYING THE MEASUREMENT FIELD FOR AN EXPOSURE CONTROL

(71) Applicant: Siemens Healthineers AG, Forchheim (DE)

(72) Inventors: Felix Denzinger, Herzogenaurach (DE); Marcus Pfister, Bubenreuth (DE); Philipp Roser, Erlangen (DE); Michael Wiets, Langensendelbach (DE)

(21) Appl. No.: 19/207,762

(22) Filed: May 14, 2025

(30) Foreign Application Priority Data
May 17, 2024 (DE) 10 2024 204 576.4

(51) Int. Cl. A61B 6/08; A61B 6/00; A61B 6/50

(52) U.S. Cl. CPC A61B 6/08; A61B 6/527; A61B 6/542; A61B 6/4441; A61B 6/504

(57) ABSTRACT

A computer-implemented method for specifying the measurement field for an exposure control of an X-ray facility includes: receiving an X-ray image by a computer unit for exposure control; recognizing at least one device marker in the X-ray image; establishing an image shape of the at least one device marker; establishing an image position of the at least one device marker; specifying the position and the shape and/or size of the measurement field dependent upon the image position and the image shape of the at least one device marker; and providing measurement field parameter values that indicate the position, the shape, and/or the size of the measurement field.

The present patent document claims the benefit of German Patent Application No. 10 2024 204 576.4, filed May 17, 2024, which is hereby incorporated by reference in its entirety.

### TECHNICAL FIELD

The disclosure relates to a method for specifying the measurement field for an exposure control, and an X-ray facility configured for carrying out such a method and a corresponding computer program product and a corresponding computer-readable storage medium.

### BACKGROUND

X-ray systems, (e.g., angiography systems), may have an automatic exposure control for X-ray recordings that are intended to provide a constant or optimum image quality at a lowest possible X-ray dose to the patient and the medical personnel. The exposure control controls X-ray exposure parameters, for example, the voltage and current of the X-ray tube. The exposure control may also control a spatial delimitation or a filtration of the X-ray beam through a collimator or through a filter, for example, a wedge filter.

As an input variable for the exposure control, an image quality value according to an image quality measure may be established in a particular region of the X-ray detector. This region of the X-ray detector is designated the measurement field. The measurement field may be defined in advance for different X-ray recording situations in different simple geometrical forms, for example, as an ellipsoid, a rectangle, or a combination thereof.

There are X-ray measuring situations in which small or complex-shaped image regions of interest (ROI) occur, for example, with stenoses or aneurysms of the blood vessels. If a generic measurement field is used, in many situations this is not configured to such image regions. As a consequence, the image quality in the image region of interest is possibly not optimal since interfering regions outside this image region, which however lie within the measurement field, influence the exposure control.

This problem is heightened if movements occur in the X-ray image, for example, heart or breathing movements of the patient.

### SUMMARY AND DESCRIPTION

The disclosure addresses these problems by way of a method, an X-ray facility, a computer program product, and a computer-readable storage medium as described herein.

The scope of the present disclosure is defined solely by the appended claims and is not affected to any degree by the statements within this summary. The present embodiments may obviate one or more of the drawbacks or limitations in the related art.

The disclosure proposes adapting the measurement field with the aid of specifically constructed radiopaque device markers. The term device is intended to mean instruments, implants, and apparatuses that are mapped in the X-ray image. The term specific construction is intended to mean, in particular, the shape, size, and number of the markers of a specific device. The term device marker is thus intended to mean X-ray markers present on or applied to a device. Such X-ray markers may be specially applied to a device, for example, as a coating of a radiopaque material. However, they may also be formed by a radiopaque structure in the device, for example, by way of radiopaque construction elements of the device or by way of radiopaque material compositions of elements of the device.

A method according to the disclosure includes the following method acts.

In one act, the device marker(s) of interest visible in the X-ray image are acquired. Which device markers are of interest and how they are represented in the X-ray image is predetermined dependent upon the method that is to be carried out. The devices that are marked in a radiopaque manner may be flow diverters, stents, shunts, thrombectomy devices such as aspiration catheters or stent retrievers, guide wires, balloon catheters, surgical instruments, biopsy needles, ablation needles, coils, or other devices such as implanted pacemakers or cardioverters. Based upon the specific construction of the radiopaque markers, it may be recognizable, for example, which device the marked device is in each case. Depending upon the method to be carried out, the radiopaque device markers of particular devices or particular types of devices may be treated as prioritized in comparison with the device markers of particular other devices or types of device. For example, device markers of devices for treatment such as flow diverters may be treated as prioritized over device markers of devices for navigation, such as guide wires. Optionally, all markers may first be acquired in the X-ray image before the device markers of interest are then identified.

In an additional act, the measurement field is specified with the aid of the specifically configured device marker of interest. For example, a single device marker may indicate, by way of its shape or size, the measurement field size that is to be set. For example, a single device marker may indicate, by way of its shape, the direction in which the measurement field is to extend starting from the device marker. For example, a plurality of device markers may indicate the periphery and/or the outer boundary of the measurement field that is to be set. For example, a plurality of device markers may each indicate the mid-point of a plurality of individual image regions of predetermined shape and size and assemble the measurement field that is to be set from the unifying of the plurality of image regions. The measurement field is used by the exposure control of the X-ray facility in order to adapt the exposure as well as possible to the respective prevailing situation. In other words, the measurement field is used by the exposure control for controlling the X-ray facility.

Optionally, the measurement field that is to be set may additionally be delimited by collimators and filters, for example, wedge filters. By way of a collimation, primarily, a dose saving may be achieved. In addition, scattered radiation may be reduced. A contrast improvement may thus be achieved, which represents an improvement in the image quality. Finally, in this way, the collimation may also contribute to the optimization of the exposure control.

According to the disclosure, a computer-implemented method for specifying a measurement field for the exposure control of an X-ray facility includes the following acts.

In act S1, an X-ray image is received by a computer unit for exposure control.

In act S2, at least one device marker in the X-ray image is recognized.

In act S3, an image shape of the at least one device marker is established.

In act S4, an image position of the at least one device marker is established.

In act S8, the position and the shape and/or size of the measurement field is specified dependent upon the image position and the image shape of the at least one device marker.

In act S9, measurement field parameter values are provided that indicate the position, shape and/or size of the measurement field.

The radiopaque device marker imparts an item of image information regarding the device or the type of device and the image position of the device. Advantageously, the image information may be derived directly and without delay at any time from the X-ray image, so that the image information relates in real time, at any time, to the current situation that is mapped in the X-ray image. By specifying the measurement field dependent upon the device marker(s), it is therefore also configured to the device or the type of device or the image position of the device. Therefore, in the specifying of the measurement field, properties of the device or intended uses of the device are taken into account. In addition, collimator and filter settings may also be adapted dynamically.

If the device in question has, for example, a movement direction, the position of the measurement field may take account of this direction. If the device in question has, for example, an action radius or an effective range, the size of the measurement field may take account of this action radius or effective range. The effective range may be relevant, for example, for ablation by ablation needles. The effective range may be relevant, for example, in the context of embolization or contrast enhancement by contrast medium. The action radius may be relevant for catheters in the coronary arteries, for example, balloon catheters with balloon markers that move together with the heart and breathing motion accordingly. The action radius may thus be defined by the anatomy and physiology or may be determined on the basis thereof.

According to an advantageous embodiment of the method, in act S2, at least two device markers are recognized and, in act S8, the specification of the position and the shape and/or size of the measurement field takes place dependent upon the respective image positions and/or the respective image shapes of the at least two device markers.

Advantageously, if the measurement field is specified dependent upon two or more device markers, the device markers may thereby indicate, for example, the desired size of the measurement field or the desired shape of the measurement field in that they indicate the respective boundary of the measurement field. The specification of the measurement field by indicating its boundaries by the device markers is a particularly simple and uncomplicated embodiment. For this purpose, it is sufficient to follow the position of the device markers once they have initially been identified as device markers of interest. Following the position of the device markers is then easily possible without having to recognize its image shape continually. In order to track the image position, as compared with the recognition of the image shape of the device markers, a lower X-ray dose may advantageously also be sufficient. Therein, the device markers may directly indicate the respective boundary of the measurement field directly or the respective boundary may be specified at a predetermined spacing from the device markers. In this way, the region between the device markers is incorporated in an uncomplicated manner into the measurement field. This is advantageous, in particular, if the intervention or use of the devices marked with the device markers is related to, and/or takes place in, this region. By way of the specification of the measurement field to this region, it is then provided that the exposure control is optimized, in particular, for this region.

According to an advantageous embodiment of the method, the method described above includes the further act S5 according to which the at least one device marker is assigned, in dependency upon its image shape, to a class of device markers, and wherein, in act S8, the specification of the position and the shape and/or size of the measurement field takes place in additional dependency upon the assigned class.

By taking account of the class of the device marker(s), the method may be adapted in a particularly flexible manner to different situations and device constellations. This adaptation may advantageously be carried out alone based upon the respective image information if information relating to the classes of device markers and their respective significance to the shape and size of the measurement field are available. This information may be capable of being retrieved, for example, from a database or an allocation table. Classes of device markers may be specified, for example, on the basis of the application purpose of the devices provided with the device markers. For example, there may be classes for balloon catheter treatment in the heart, for balloon catheter treatment in peripheral blood vessels, for thrombectomy in the heart, for thrombectomy in the brain, etc. Classes of device markers may be specified, for example, on the basis of the type of devices provided with the device markers. Classes may exist, for example, for balloon catheters, for aspiration catheters, for stent retriever catheters, for guide wires, for pressure measuring catheters, etc. Classes of device markers may be specified, for example, on the basis of the expected combination of further devices having the devices provided with the device markers. The combinations may be combinations integrated with one another, so-called device stacks or combinations of separate devices. For example, there may be classes for the expected combination with a further device, with two further devices, with three further devices, etc.

In the following, a series of possible configurations and uses of device markers for carrying out a method is set out.

The measurement field may be determined on the basis of the device markers at the start and the end of a device of interest.

The measurement field may be a simple geometrical form, for example, a rectangle, that is anchored by the device marker of interest. If there are, for example, two device markers of interest, these may indicate two opposite edges of a rectangular measurement field.

The measurement field may be determined on the basis of the device markers of a plurality of devices. For example, an anchor of the measurement field may be determined on the basis of the device marker of a catheter and another anchor of the measurement field on the basis of the device marker furthest removed from the device marker of the catheter.

Specific device markers may indicate individual devices. For example, the size or the number or the size and number of the device markers may indicate an intended sequence of the arrangement of devices for an intended use, for example, a sequence of the arrangement from proximal to distal.

A map or a model of a blood vessel structure or of a hollow organ may be used to further determine the measurement field. For example, exclusively a structure within the region of interest indicated by the device marker and additionally within the blood vessel structure or the hollow organ may be considered to be a measurement field. The map or the model of the vessel structure or of the hollow organ may be based upon a 2D-DSA recording, a 2D road map, a 3D road map, or a 3D-DSA recording, or upon a plurality of these possibilities.

A safety margin may be used, about which the measurement field is to be enlarged. The device markers may indicate how large the safety margin may be selected. Changes to the safety margin may be enabled in that a user interface enables a corresponding user input. The user input related to the safety margin may then additionally be taken into account on specification of the measurement field, for example in that the safety margin is enlarged or reduced by a value according to the user input.

The image parameters may be adapted automatically to the content of the region of interest acquired with the aid of device markers.

The method may advantageously be applied not only for static image contents, but rather also for dynamic image contents. On the basis of the device markers, movements of the devices may be followed automatically. In this way, the measurement field may be adapted dynamically. In addition, collimator and filter settings may also be adapted dynamically.

According to an advantageous embodiment of the method, it is therefore proposed, for the event of non-static image contents, to determine the measurement field with the aid of radiopaque device markers in combination with a movement model for their movement.

If radiopaque device markers are used exclusively statically, the measurement field may only be determined retrospectively in each case, based upon a respective preceding X-ray image. If larger or more rapid movements occur, for example, in the case of stents in the coronary artery due to the heart movement, the retrospective determination of the measurement field may be insufficient. For example, the radiopaque device markers may move together with the device, partially or entirely out of the measurement field, due to heart or breathing movements or a combination of both. For example, the radiopaque device markers may additionally even move out of the spatial region of the X-ray radiation, in particular, if it is spatially delimited by collimation.

In order to solve this problem, in an advantageous embodiment of the method, it is proposed to predict the position of the device marker in the X-ray image given dynamic image contents, on the basis of a movement model.

For this purpose, in one act, a calibration of a movement model is initially carried out. The movements coming into consideration may be cyclical and/or periodic and may be caused by the heartbeat or by breathing. In cases of cyclical movements, the calibration of the movement model may, for example, include a single heart phase, for example, confirmed by an ECG or an individual breathing phase, for example, by way of a chest belt or a ventilation device. The calibration phase may also include a predefined timeframe within which highly probably a complete heart or breathing phase is run through. The calibration phase may also include a timeframe that results from a combination of a plurality of parameters. An initial movement model may also be generated via the respective current ECG information and the associated position of the X-ray marker(s). Therein, the respective projection direction of the X-ray facility may also be taken into account. In addition, prior knowledge of the movement pattern of the X-ray marker(s) may additionally be derived.

In the calibration phase, a movement model is generated that describes the most probable sequence of positions of the device markers in the X-ray image. On the basis of the movement model, the respective most likely subsequent position of a device marker relative to a respective prior position is specified.

During the calibration, initially, all the radiopaque device markers of interest in the X-ray image are acquired. Optionally, at first all radiopaque device markers are acquired and subsequently, the device markers of interest are identified. Then the positions of the device markers of interest in the X-ray image are followed in chronological sequence during the calibration phase, which may include a heart cycle or a breathing cycle. From the chronological sequence of the positions of the device markers, a movement model is then generated. The movement model may be generated, for example, as a function over time or as a function dependent upon additionally acquired sensor data. The additionally acquired sensor data may relate for example to a heart cycle, for example, ECG data, or a breathing cycle, for example, chest belt data.

In a further act, the calibrated movement model is used in order to determine a measurement field for the subsequent X-ray images (also called frames). Optionally, the measurement field may additionally be framed by automatic adaptation of collimators or filters or semi-transparent wedge filters in order to reduce the dose delivered to the patient.

According to an advantageous embodiment, the method may further include the following acts. In act S6, a movement model is obtained by way of the computer unit for exposure control, wherein the movement model defines a chronological sequence of a movement of a device marker. Further, in act S7, an image position and/or image shape of the device marker to be expected is predicted, following a current image position and/or image shape of the at least one device marker, with the aid of a movement model, wherein, in act S8, the specification of the position and the shape and/or size of the measurement field takes place dependent upon the image position and/or image shape of the device marker that are to be expected.

Advantageously, during the dynamic determination of the measurement field with the aid of device markers and their movement model, a smaller safety margin is sufficient since the movement model predicts the future positions of the device marker and thus of the measurement field. The prediction on the basis of the movement model reduces the risk that device markers together with the respective device move out of the measurement field partially or altogether due to heart or breathing movements or a combination of both.

Advantageously, due to a smaller safety margin when the measurement field is additionally framed by automatic adaptation of collimators or filters or semi-transparent wedge filters, the X-ray dose may be reduced by the spatial delimitation or filtration of the X-ray beam.

According to an advantageous embodiment of the method, the image positions and/or image shapes of the at least one device marker that are to be expected as predicted by the movement model are compared with the actually occurring image positions and/or image shapes. Deviations exceeding a predetermined threshold value are then used in order to adapt the movement model on the basis of the actually occurring image positions and/or image shapes. By this, the movement model may be presently configured to the actual conditions and advantageously the accuracy of the predictions may be improved. By this, a generic non-individual movement model may also be used and advantageously configured to the individual case, in other words, it may thus be individually adapted and/or individualized.

In an advantageous embodiment of the method, a direction and/or a distance of the change of the image position of the at least one device marker between the current image position and the expected image position is established. Then, in act S8, the specification of the position and the shape and/or size of the measurement field takes place dependent upon the direction and/or distance of the change.

Thus, a geometrical size that is simple and uncomplicated to establish for a prediction of an expected future image position of the device marker(s) is available. In one embodiment, the position of the measurement field may then be specified on the basis of the predicted image position.

In an advantageous embodiment of the method, a direction and/or a distance of the change of the image positions of at least two device markers between their respective current image position and their respective expected image position is established. In act S8, the specification of the position and the shape and/or size of the measurement field then takes place dependent upon the directions and/or distances of the changes.

If the measurement field is specified dependent upon two or more device markers, the device markers may thereby indicate, for example, the desired size of the measurement field or the desired shape of the measurement field in that they indicate the respective boundary of the measurement field. Therein, they may directly indicate the respective boundary or the respective boundary may be specified at a predetermined spacing from the device markers. This is advantageous in particular if the intervention or the use of the devices marked with the device markers is to relate to this region. By way of the specification of the measurement field to this region, it is then provided that the exposure control is optimized for this region. By additionally taking account of the movement model, positions for the two or more device markers at which the positions of the device markers change relative to one another may be predicted. By this, for example, situations may be taken into account in which the device markers change their positions relative to one another due to a change in the positions of the devices. This may occur in particular if the devices are moved with one another or with their respective environment in the context of an intended use of the devices. Through the prediction of the positions of the device markers, thereby, in particular, required changes in the size of the measurement field may be predicted.

An expected future image shape of the device marker(s) may also be called upon as the basis for the determination of the measurement field. In an embodiment, a device marker points, by virtue of its image shape, in a direction in which the measurement field is to be situated. The predicted image shape may therein point in a different direction from the image shape of a device marker in the preceding image, if the device rotates, for example, from image to image. Thus, the measurement field may be specified according to the predicted direction pointed to by the device marker.

In an advantageous embodiment of the method, a direction and/or a distance of the change of the image positions of at least two device markers between their respective current image position and their respective expected image position is established. In act S8, the specification of the position and the shape and/or size of the measurement field then takes place dependent upon the directions and/or distances of the changes.

In the specification of the measurement field, regardless of whether a movement model is called upon or not, a feature recognized in the X-ray image may also be taken into account. The feature may be an anatomical feature. The device marker(s) may be provided on a device that relates in a predetermined manner to the anatomical feature. The device may be a catheter and the anatomical feature may be a blood vessel. The catheter may be intended to be situated in the blood vessel. In this exemplary case, when the measurement field is specified, it may be taken into account that the course of the blood vessel may be within the measurement field as far as possible.

Further embodiments and advantages are disclosed in the dependent claims and the following description of exemplary embodiments making reference to the drawings.

### BRIEF DESCRIPTION OF THE DRAWINGS

FIG. 1 depicts an example of an X-ray facility configured to carry out the method.

FIG. 2 depicts an example of a method without a movement model.

FIG. 3 depicts an example of a method with a movement model.

FIG. 4 depicts an example of an individual device marker indicating a direction.

FIG. 5 depicts an example of two cooperating device markers.

FIG. 6 depicts an example of two mutually complementary device markers.

FIG. 7 depicts an example of a device marker together with the movement according to the movement model.

FIG. 8 depicts an example of a device marker together with the change according to the movement model.

FIG. 9 depicts an example of a device marker and additional image feature.

### DETAILED DESCRIPTION

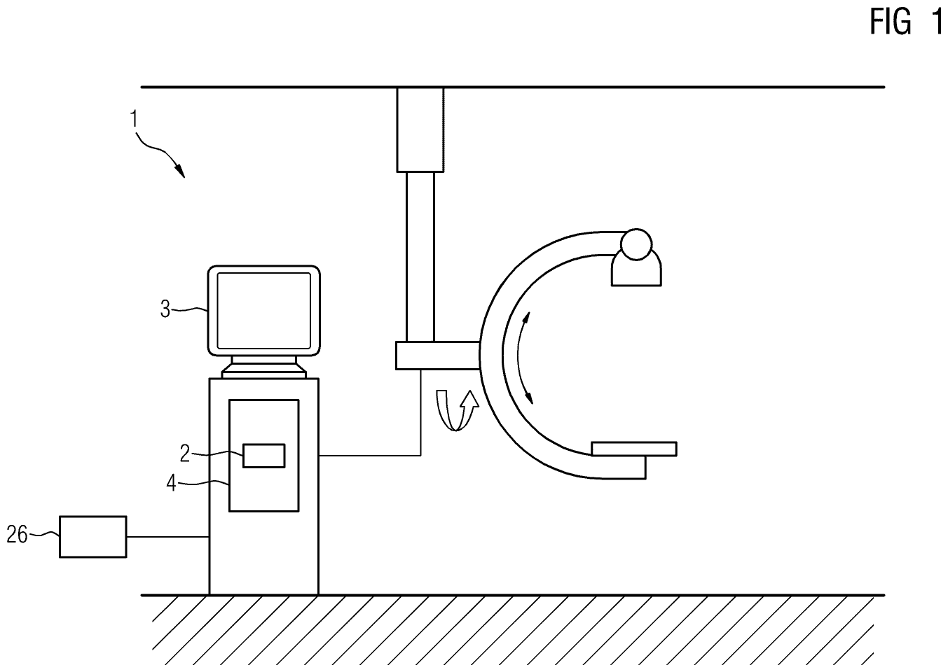

FIG. 1 shows schematically an X-ray facility 1 configured to carry out the disclosure. The X-ray facility 1 includes a ceiling-mounted C-arm with an X-ray source and an X-ray detector. The C-arm is connected to a control unit 4 by way of which the X-ray facility 1 is controlled.

The control unit 4 includes a computer unit 2 by way of which the exposure control is carried out. In particular, by way of the computer unit 2, the measurement field for the exposure control is specified. The computer unit 2 may be integrated, for example, into the control unit 4. The computer unit 2 may also be formed, for example, by a component additional to the control unit 4. It is based upon a microprocessor together with a working memory store that may both be configured in a known manner.

The control unit 4 is also connected to an external data source 26. The external data source 26 may be configured for carrying out an embodiment. For this purpose, it may keep data available that includes information relating, for example, to device markers, the image shape of device markers, classes of device markers, the shape and size of measurement fields, devices, procedures to be carried out with the devices, anatomical features relating to device markers, or suchlike. The data may be present, for example, in the form of a database or table. The data reveals, for example, what shape of a measurement field is associated with which device markers recognizable from its image shape, or what size a measurement field may have for which image shape and/or constellation of device markers, or which image regions may be included by the measurement field given the presence of an anatomical feature.

The X-ray facility 1 further includes a screen 3 for displaying X-ray images or control information.

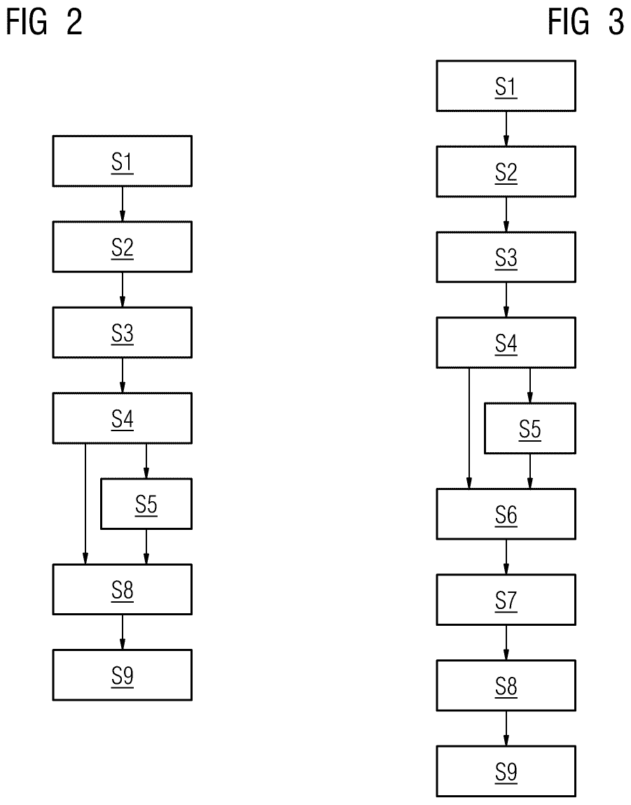

In FIG. 2, the sequence of a method is displayed schematically in individual acts, wherein the method does not use a movement model.

In act S1, the computer unit 2 receives an X-ray image. The X-ray image may have been recorded currently by the X-ray facility 1 or retrieved from a data store. For carrying out of the method, only one or more device markers are mapped in the X-ray image. The device marker(s) may be radiopaque markings that are applied to a device. The device marker(s) may also be, for example, radiopaque components that are part of the device or of which the device consists or is assembled. Device may mean, for example, a guide wire or a catheter.

In act S2, a recognition of device markers in the X-ray image takes place. Device markers may be recognized in the X-ray image by their image shape, that is, the shape represented in the X-ray image. Device markers may be configured, in known manner, as radiopaque points, groups of points, lines, groups of lines, geometric patterns such as crosses or arrows, legible characters such as digits or letters or suchlike.

The recognition of device markers may take place, for example, in two acts. In a first act, all the device markers represented in the X-ray image are recognized. Thereafter, in a second act, the device markers of interest contained within the totality of device markers are identified. The recognition of device markers may take place, for example, immediately, in that device markers of interest represented in the X-ray image are recognized immediately and directly. In order to recognize device markers in the X-ray image, standard methods of image analysis or pattern recognition may be used. The recognition may also take place, for example, with the aid of trained software.

Device markers of interest may be recognizable in that they are arranged on a particular device. Device markers of interest may be recognizable, for example, from their image shape. The image shape that device markers of interest have may be predetermined, for example, in advance. For example, device markers of interest may be cruciform, circular, or configured as an arrow. The image shape that device markers of interest have may also be specifically specified for a particular performance of the method. For example, for a particular type of method, a particular type of device marker may be predetermined in each case, for example, cruciform device markers for balloon catheter methods and circular device markers for pressure measuring catheter methods. Furthermore, the image shape of device markers may also be specified specifically for a particular type of device, for example, cruciform for balloon catheters and arrow-like for guide wires. Not least, device markers of interest may also be recognized with the aid of trained software.

The information regarding which image shape is predetermined for device markers of interest may be retrieved, for example, from a database. The information regarding which image shape is predetermined for device markers of interest may also be established, for example, on the basis of a user input. In one case, the image shape for device markers of interest may also be predetermined as standard, so that when no other information is received, in principle, the standard predetermined image shape is used.

In act S3, at least one image shape of at least one device marker of interest is established. The image shape of the device marker corresponds to the pictorial representation of the device marker in the X-ray image. The image shape may correspond, for example, to a cross, an arrow, an angle, or some other pattern.

In act S4, at least one image position of at least one device marker of interest is established. In a two-dimensional X-ray image, the image position is given by the image coordinates of the pixels, which represent the device markers in the X-ray image. If the device marker is displayed as circular, as the image position, for example, the mid-point of the circle may be called upon. For arbitrary image shapes of device markers, as the image position, for example, the geometric mid-point of the image shape may be called upon. For specifically configured device markers, as the image position, for example, a particular point of the image shape may also be called upon, for example, with an arrow-like device marker, the tip of the arrow, or with a cruciform device marker, at the crossing point.

Optionally, act S5 may follow, wherein one or more device markers are assigned to a respective class of device markers, dependent upon their particular image shape. Therein, for example, a class of device markers may be identified by way of their circular image shape, a further class of device markers by their cruciform image shape, a further class of device markers by a particular arrangement of separate individual device marker elements, etc. The classes of device markers and their image shape may be capable of being retrieved, for example, from a database or a table, or they may be obtained by way of a user input or they may be predetermined. The information regarding classes of device markers, in particular, if they are available in the form of a database or table, may be capable of being retrieved, for example, from the data source 26. For this purpose, the recognized image shape of the device marker may be used, for example, as a search criterion. In the event of a query regarding a class, the data source 26 may output, for example, the shape of the measurement field that is to be specified and is associated with this class, or details regarding the positioning of the measurement field or details regarding the size of the measurement field.

In act S8, dependent upon one or more previously recognized device markers of interest, the measurement field is specified for the exposure control. The previously established image position of the device marker and, in addition, the previously established image shape of the device marker is taken as the basis for the specification of the measurement field.

The image position of the device marker(s) is retrieved for the specification of the position of the measurement field. In addition, the image shape of the device marker(s) is taken into account for the specification of the position of the measurement field.

For example, a device marker may be configured such that its image shape indicates a direction in which the measurement field is to extend. In this case, the measurement field will therefore not be positioned centered relative to the device marker, but rather displaced relative thereto.

For example, two or more device markers may be configured such that together they span the required measurement field. The two or more device markers may be configured such that they indicate the edges or corners of a measurement field, wherein device markers may indicate edges in line form and device markers may indicate corners in an angled form. Furthermore, for example, the device markers may be configured such that, with their image shape, they indicate in which direction the measurement field extends. For example, in the case of an angular device marker, the measurement field may be extended in the direction of the enclosed angle.

For the specification of the shape of the measurement field, with a single device marker, its image shape may be called upon and with a plurality of device markers, alternatively or additionally, their spatial position to one another may be called upon.

For example, a single device marker in the X-ray image may be displayed as circular and the circle shape may indicate that the measurement field may have a round shape. For example, a single device marker in the X-ray image may be displayed as cruciform and the cross shape may indicate that the measurement field may have a rectangular shape.

For example, two or more device markers in the X-ray image may each individually indicate the image position and possibly the shape of an associated single measurement field, and the measurement field that is to be specified may be specified by combining all the individual measurement fields into an overall combined measurement field.

For example, a single device marker may indicate with its image shape or its size what size the measurement field is to have. For example, two or more device markers may indicate, by way of the spatial constellation of their representations in the X-ray image and possibly additionally their respective image shape, what size the measurement field is to have.

If, before the execution of act S8, the optional act S5 has been executed and device markers have been assigned to respective classes, each class may be taken into account in the specification of position, shape and/or size of the measurement field. Therein, it may be defined, for example, according to a predetermined rule for each class of device markers that the measurement field may have a predetermined shape, for example rectangular or round. The rule as to which position, shape, and/or size of the measurement field is assigned to a respective class of device markers may be capable of being retrieved, for example from a database.

In act S9, parameter values that describe the previously specified measurement field are provided. The parameter values describe the position, shape and/or size of the measurement field in relation to the X-ray image.

In FIG. 3, the sequence of a method is displayed schematically in individual acts, wherein the method additionally uses a movement model for the method described above in relation to FIG. 2. In this respect, the preceding explanations apply to the previously described acts of the method.

In act S6, in addition to the method described above, subsequently to act S4) and/or the optional act S5), by way of the computer unit 2, a movement model is obtained that describes a temporal sequence of a movement of one or more device markers. Therein, the computer unit 2 may establish the movement model itself, for example, on the basis of the current and previous X-ray images. The computer unit 2 may obtain the movement model, for example, from another component of the X-ray facility 1. The computer unit 2 may obtain the movement model, for example, from a database or another source. The movement model may therein be established and represented in a conventional known manner.

The movement model enables, on the basis of a previous X-ray image in each case, a prediction of a respective subsequent X-ray image. The movement model enables, on the basis of the image position and the image shape of a device marker in a preceding X-ray image in each case, a prediction for the image position and image shape of the device marker in a subsequent X-ray image in each case. The prediction provides the future image position and image shape of the device marker that are considered the most likely by the movement model. Such predictions are readily possible, for example, for identically shaped movements or cyclically recurring movements. Cyclical movements may be caused, for example, by a heartbeat or breathing activity.

In act S7, with the aid of the movement model, the image position and/or image shape of the device marker that are to be expected starting from a current image position and/or image shape are predicted. The specification of the position, shape, and/or size of the measurement field then takes place dependent upon the image position and/or image shape of the device marker that are to be expected. For example, the measurement field may be specified in the manner described above, although it is not the current, but rather the predicted image position and/or image shape of the device marker(s) that is/are used.

For example, the change in the image position of a device marker that results on the basis of the prediction may be analyzed regarding its direction. Then, the current measurement field, for example, in the current shape and size may be displaced according to the established direction, or it may be enlarged in the established direction. For example, the change in the image position of the device marker may be analyzed regarding its distance. Then the current measurement field may be enlarged according to the established distance. For example, both the direction and also the distance of the change in the image position of the device marker may be analyzed. The current measurement field may then be specified with regard to position, size, and/or shape dependent both upon direction and also distance, that is, the movement vector, of the image position of the device marker.

For example, the change in the image shape of a device marker that results on the basis of the movement model may be analyzed. Based upon the change in the image shape, a corresponding change in the measurement field may be specified. For example, a device marker that is changed in its image shape may point in a changed direction in the X-ray image. Then, the position of the measurement field may be oriented to the changed direction. For example, a device marker that is changed in its image shape may have a larger or smaller spatial extent. Then, for example, an enlarged or reduced measurement field dependent thereon may be specified.

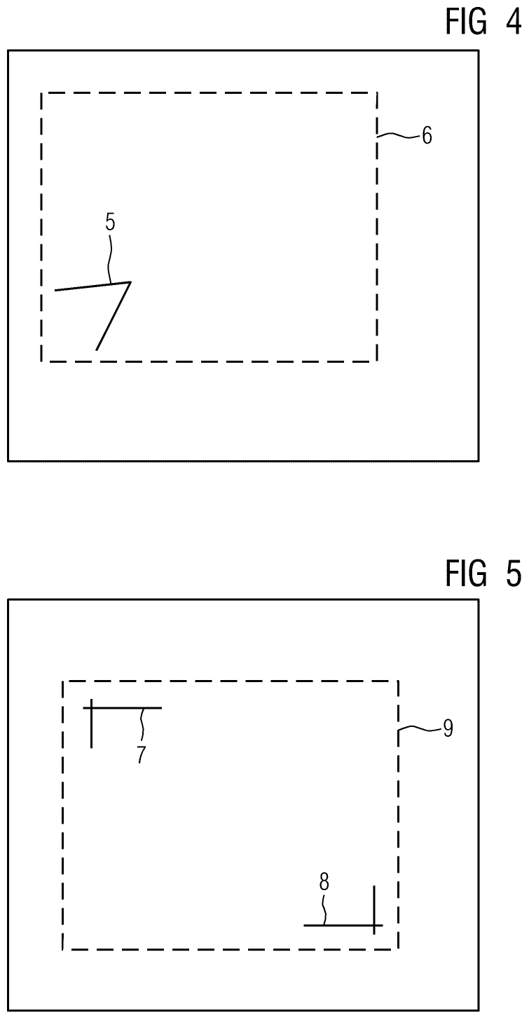

FIG. 4 shows schematically an example of a device marker 5. The device marker 5 has an arrow-like image shape and thus points in a direction, up and to the right in the drawing. According to the method described above, the rectangular measurement field 6 is positioned, dependent upon the image shape of the device marker 5, such that it includes the image region in the direction indicated.

FIG. 5 shows schematically an example of two individual device markers 7, 8. The device markers 7, 8 have an angled image shape. Therein, the respective right angles that are included each point toward the measurement field. Furthermore, each right angle that is included also points in the direction in which the respective other device marker 7, 8 is situated. Thus, proceeding in each case from one of the device markers 7, 8, it may be seen where the other device marker 7, 8 may be situated. According to the method described above, the rectangular measurement field 9 is positioned, dependent upon the respective image shape of the device markers 7, 8, such that it includes the indicated image region enclosed by the device markers 7, 8.

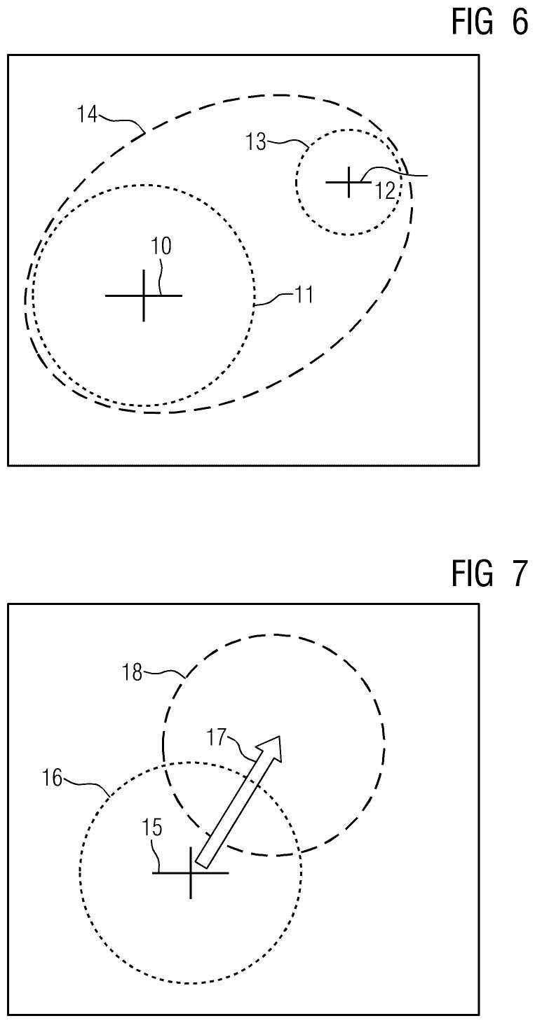

FIG. 6 shows an example of two individual device markers 10, 12. The device markers 10, 12 have a cruciform image shape. According to the method described above, a larger individual measurement field 11 that is circular is assigned to the device marker 10 with the larger image shape. A smaller individual measurement field 13, that is also circular, is assigned to the device marker 12 with the smaller image shape. Based upon these individual measurement fields 11, 13, the measurement field 14 is specified such that it includes the individual measurement fields 11, 13. In the example shown, the measurement field 14 is specified in a round shape based upon the circular shape in the individual measurement fields 11, 13. Alternatively, however, the measurement field 14 may also be given a rectangular shape. The measurement field 14 at least encloses the individual measurement fields 11, 13.

FIG. 7 shows an example of a device marker 15 for which a movement illustrated by the arrow 17 has been established. According to the method described above, for this purpose, a movement model for the device marker 15 has been obtained. Based upon the movement model, starting from the current image position of the device marker 15 in the X-ray image that is illustrated in the mapping, the most probable image position for the subsequent X-ray image is predicted. For the current image position of the device marker 15, the measurement field 16 would need to be specified. However, the image position predicted by the movement model for the specification of the measurement field 18 is called upon, so that the measurement field 18 is displaced relative to the measurement field 16 according to the predicted movement of the device marker 15.

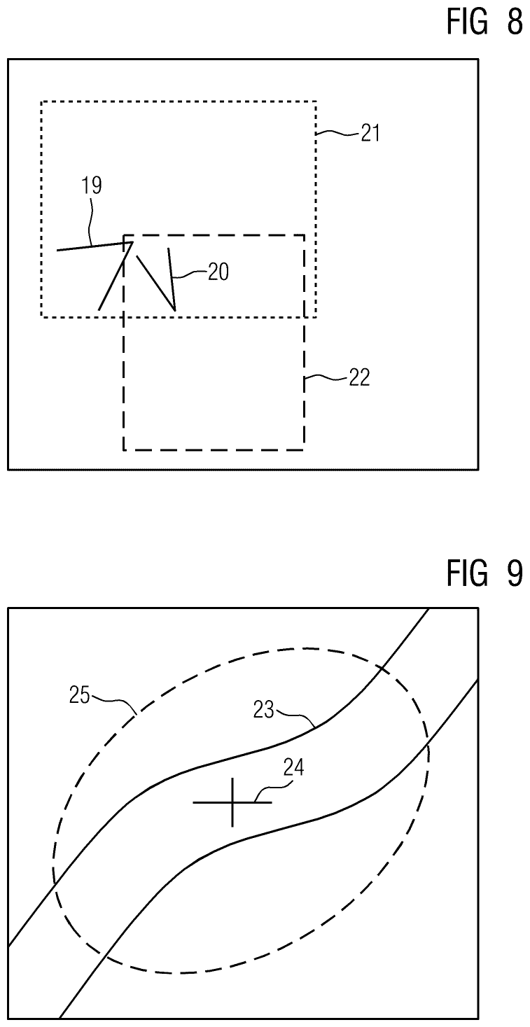

FIG. 8 shows an example of one individual device marker 19. The device marker 19 has an arrow-like image shape. A measurement field 21 is associated with the device marker 19. The measurement field 21 includes the image region in the direction of which the arrow-like device marker 19 points. According to the method described above, a movement model has been obtained for the device marker 19. The movement model proceeds from the current image shape of the device marker 19 in the X-ray image, as shown in the mapping, and predicts the most probable image shape of the device marker 20 for the subsequent X-ray image. The predicted image shape of the device marker 20 is both rotated relative to the current image shape of the device marker 19 and is also narrower, where narrower is used to mean that the arrow-like image shape is more pointed. According to the changed direction in which the predicted device marker 20 points as compared with the current device marker 19, the position of the measurement field 22 is changed such that it encompasses the changed image region to which the device marker 20 points. According to the narrower image shape of the predicted device marker 20, the measurement field 22 is narrower and/or smaller than the measurement field 21.

FIG. 9 shows an example of a device marker 22 which has a cruciform image shape. The course of a blood vessel 23 is also shown schematically. The device marker 22 belongs to a device, for example, a catheter situated in the blood vessel 23. The blood vessel 23 is recognized according to the method described above, as an anatomical feature. The measurement field 25 is specified dependent upon this anatomical feature such that it encloses it as far as possible. Therefore, the measurement field 25 is positioned in the mapping such that its longitudinal axis is oriented parallel to the course of the blood vessel 23.

Anatomical features or types of anatomical feature may be capable of being retrieved, for example, from a database or a table, or they may be obtained by way of a user input or they may be predetermined. The information relating to anatomical features, in particular, if it is available in the form of a database or table, may be capable of being retrieved, for example, from the data source 26. For this purpose, for example, the recognized anatomical feature may be used as a search criterion. In the case of a query regarding an anatomical feature, the data source 26 may output, for example, the shape of the measurement field that is to be specified and is associated with this feature or this feature type, or details regarding the positioning of the measurement field or details regarding the size of the measurement field.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for specifying a measurement field for exposure control of an X-ray facility, the method comprising:
receiving an X-ray image by a computer unit for the exposure control;
recognizing at least one device marker in the X-ray image;
establishing an image shape of the at least one device marker;
establishing an image position of the at least one device marker;
specifying a position and a shape and/or a size of the measurement field dependent upon the image position and the image shape of the at least one device marker; and
providing measurement field parameter values that indicate the position and the shape and/or the size of the measurement field.

2. The method of claim 1, wherein the recognizing of the at least one device marker comprises recognizing at least two device markers, and
wherein the specifying of the position and the shape and/or the size of the measurement field takes place dependent upon respective image positions and/or respective image shapes of the at least two device markers.

3. The method of claim 1, further comprising:
assigning the at least one device marker, dependent upon the image shape of the at least one device marker, to a class of device markers to provide an assigned class,
wherein the specifying of the position and the shape and/or the size of the measurement field is additionally dependent upon the assigned class.

4. The method of claim 3, further comprising:
obtaining a movement model by way of the computer unit for the exposure control, wherein the movement model describes a chronological sequence of a movement of the at least one device marker; and
predicting an image position and/or an image shape of the at least one device marker to be expected following a current image position and/or a current image shape of the at least one device marker using the movement model to provide a predicted image position and/or a predicted image shape,
wherein the specifying of the position and the shape and/or the size of the measurement field is additionally dependent upon the predicted image position and/or the predicted image shape.

5. The method of claim 4, further comprising:
recognizing an anatomical feature in the X-ray image,
wherein the specifying of the position and shape and/or the size of the measurement field is additionally dependent upon the anatomical feature.

6. The method of claim 1, further comprising:
obtaining a movement model by way of the computer unit for the exposure control, wherein the movement model describes a chronological sequence of a movement of the at least one device marker; and
predicting an image position and/or an image shape of the at least one device marker to be expected following a current image position and/or a current image shape of the at least one device marker using the movement model to provide a predicted image position and/or a predicted image shape,
wherein the specifying of the position and the shape and/or the size of the measurement field is additionally dependent upon the predicted image position and/or the predicted image shape.

7. The method of claim 6, wherein the predicted image position and/or the predicted image shape of the at least one device marker are compared with an actually occurring image position and/or an actually occurring image shape of the at least one device marker, and
wherein the movement model is configured based on the actually occurring image position and/or the actually occurring image shape when a deviation lies above a predetermined threshold value.

8. The method of claim 7, wherein a direction and/or a distance of a change of the image position of the at least one device marker between the current image position and the predicted image position is established, and
wherein the specifying of the position and the shape and/or the size of the measurement field is dependent upon the direction and/or the distance of the change.

9. The method of claim 7, wherein directions and/or distances of changes of image positions of at least two device markers between respective current image positions and predicted image positions is established, and
wherein the specifying of the position and the shape and/or the size of the measurement field is dependent upon the directions and/or the distances of the changes.

10. The method of claim 1, further comprising:
recognizing an anatomical feature in the X-ray image,
wherein the specifying of the position and shape and/or the size of the measurement field is additionally dependent upon the anatomical feature.

11. An X-ray facility comprising:
a computer unit configured to:
receive an X-ray image;
recognize at least one device marker in the X-ray image;
establish an image shape of the at least one device marker;
establish an image position of the at least one device marker;
specify a position and a shape and/or a size of a measurement field for exposure control of the X-ray facility dependent upon the image position and the image shape of the at least one device marker; and
provide measurement field parameter values that indicate the position and the shape and/or the size of the measurement field.

12. A non-transitory computer-readable medium having a computer program product, the computer program product comprising program elements that, when executed by a computer unit, cause the computer unit to:
receive an X-ray image;
recognize at least one device marker in the X-ray image;
establish an image shape of the at least one device marker;
establish an image position of the at least one device marker;
specify a position and a shape and/or a size of a measurement field for exposure control of an X-ray facility dependent upon the image position and the image shape of the at least one device marker; and
provide measurement field parameter values that indicate the position and the shape and/or the size of the measurement field.
